(19) United States
(12) Patent Application Publication
(10) Pub. No.: US 2026/0250296 A1
(43) Pub. Date: Aug. 27, 2026

(54) PROCESS FOR SYNTHESIZING NAPHTHYRIDINE DERIVATIVES AND INTERMEDIATES THEREOF

(71) Applicant: AMGEN INC., Thousand Oaks, CA (US)

(72) Inventors: Matthew G. Beaver, Natick, MA (US); Sebastien Caille, Moorpark, CA (US); Charles Joseph Dooley, III, Arlington, MA (US); Hsiao-Wu Hsieh, Quincy, MA (US); Heather Claire Johnson, Jamaica Plain, MA (US); Neil Fred Langille, Sudbury, MA (US); James Murray, Camarillo, CA (US); Tessa Hamilton Trexler Myren, Westlake Village, CA (US)

(21) Appl. No.: 19/157,399

(22) PCT Filed: Feb. 28, 2024
(86) PCT No.: PCT/US24/17587

§ 371 Date: Aug. 18, 2025

Related U.S. Application Data
(60) Provisional application No. 63/487,527, filed on Feb. 28, 2023.

(51) Int. Cl. C07D 491/147; C07C 269/06; C25B 3/05; C25B 3/07; C25B 3/09; C25B 3/11; C25B 3/29

(52) U.S. Cl. CPC C07D 491/147; C07C 269/06; C25B 3/05; C25B 3/07; C25B 3/09; C25B 3/11; C25B 3/29

(57) ABSTRACT

The disclosure provides processes for a cross-coupling reaction comprising (a) exposing a mixture comprising a nickel (11) precatalyst, a phosphine, and an electrolyte in a solvent to a current to produce a nickel (0) catalyst; and (b) catalyzing the cross-coupling reaction with the nickel (0) catalyst. The disclosure also provides processes for preparing Compound (A), or a salt thereof, Compound (F), and Compound (I), as described herein.

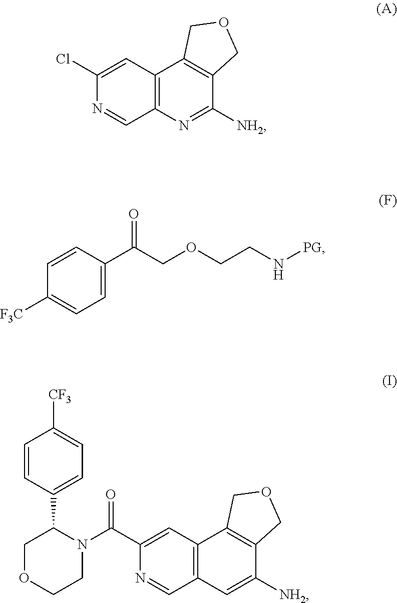

### BACKGROUND

Naphthyridine derivatives and intermediates have been shown to be important in a number of biological applications. In order to investigate their efficacy, large quantities of the materials are needed. As such, there is a need for efficient, cost-effective processes for preparing naphthyridine derivatives that are amenable to large scale.

### SUMMARY

The disclosure provides a process for a cross-coupling reaction comprising (a) exposing a mixture comprising a nickel(II) precatalyst, a phosphine, and an electrolyte in a solvent to a current to produce a nickel(0) catalyst; and (b) catalyzing the cross-coupling reaction with the nickel(0) catalyst.

The disclosure also provides a process for preparing Compound A, or a salt thereof:

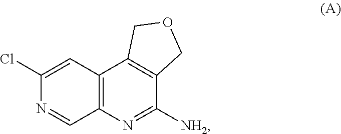 [SMILES: Nc1nc2cnc(Cl)cc2c2c1COC2]

comprising (a) preparing a nickel(0) catalyst by exposing a mixture comprising a nickel(II) precatalyst, a phosphine, and an electrolyte in a solvent to a current to produce the nickel(0) catalyst; and (b) contacting the nickel(0) catalyst with Compound B

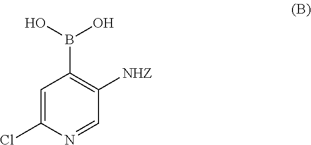

### and Compound C

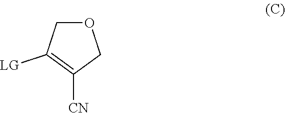

in an admixing solvent to form Compound A or a salt thereof, wherein Z is an amine protecting group and LG is a leaving group.

The disclosure further provides a process for preparing Compound F

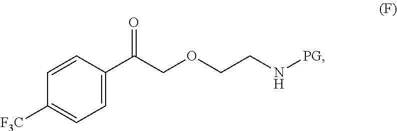

comprising (a) reacting Compound H

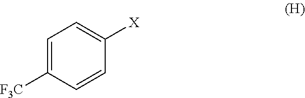

in a first solvent with an organolithium reagent to form a lithiated intermediate; and (b) reacting the lithiated intermediate with Compound G

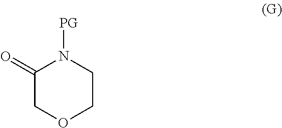

in a second solvent to form Compound F, wherein X is halogen and PG is a protecting group.

The disclosure also provides processes for preparing Compound (I)

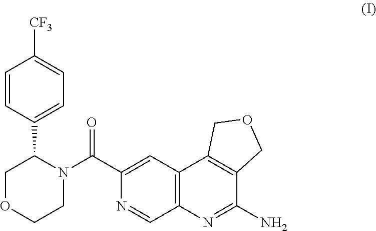 [SMILES: Nc1nc2cnc(C(=O)N3CCOC[C@@H]3c3ccc(C(F)(F)F)cc3)cc2c2c1COC2]

using the processes disclosed herein.

### DETAILED DESCRIPTION

Provided here are processes for preparing various compounds useful as active pharmaceutical ingredients (API) and/or synthetic intermediates thereof (e.g., Compound A or a salt thereof, Compound F, and Compound I, as described herein).

As such, the disclosed processes provide a decreased environmental impact (for example, improved process “greenness”) as measured by, for example, a reduction of halogenated solvents.

The disclosed processes provide several advantages over conventional processes. For example, in various embodiments, the disclosed processes provide electrochemical processes that replace more costly conventional processes that use harsh conditions (e.g., toxic metal reagents) or that require more challenging reaction conditions (e.g., air-sensitive reagents). The disclosed processes also provide a decreased environmental impact (e.g., improved process “greenness”) as measured by, for example, a reduction in waste streams. These advantages are particularly important when preparing APIs, which require stringent control of conditions to minimize toxic impurities and waste by-products, particularly when produced on a commercial scale.

In some embodiments, the disclosed processes provide an electrochemical process that replaces the use of harsh metal bases (e.g., n-butyllithium and hexylmagnesium chloride, manganese and zinc metal reductants). In some embodiments, the disclosed processes use nickel(II) containing compounds that are air-stable and less expensive than other conventional reactants.

In some embodiments, the disclosed processes are conducted using continuous manufacturing processes (i.e., “flow chemistry” or “continuous chemistry”). As used herein, continuous manufacturing refers to an integrated system of unit of operations, with constant flow (steady or periodic). The disclosed processes utilizing continuous chemistry can provide the production of gram to metric ton quantities of active pharmaceutical ingredients (APIs). In some embodiments, the disclosed processes comprise a combination of steps that are conducted using batch chemistry and steps conducted using continuous chemistry (i.e., “semi-batch mode”).

The continuous processes disclosed herein advantageously provide a reduction in reaction times and a reduction in reaction by-products (e.g., as measured by solids formation). In some embodiments, the disclosed flow processes for preparing Compound F comprise a residence time (e.g., reaction time) of less than 30 seconds, as compared to almost 3 hours in a conventional process. In addition, the continuous processes disclosed herein improve overall efficiency and operation of the synthesis. For example, in some embodiments, the continuous processes for preparing Compound F described herein replace three crystallizations and a carbon treatment in the conventional process with a single crystallization step—thereby reducing the number of operations and improving the overall yield and efficiency.

In some embodiments comprising a continuous process, the process is conducted in a continuous process apparatus. An illustrative continuous process apparatus is a plug-flow reactor.

In some embodiments, the disclosed processes are conducted in batch mode (i.e., “batch chemistry” or “fed-batch mode”).

In some embodiments, the disclosure provides processes for a cross-coupling reaction comprising (a) exposing a mixture comprising a nickel(II) precatalyst, a phosphine, and an electrolyte in a solvent to a current to produce a nickel(0) catalyst; and (b) catalyzing the cross-coupling reaction with the nickel(0) catalyst.

### Cross-Coupling Reaction

In some embodiments, the disclosed processes provide processes for cross-coupling reactions. As understood, a cross-coupling reaction occurs when two fragments (e.g., metal-containing fragment and an electrophile fragment) are joined together, typically using a metal catalyst. Cross-coupling reactions can be used to form carbon-carbon bonds or carbon-heteroatom bonds. In some cases, the metal-containing fragment and the electrophile fragment are present within the same compound (i.e., an intra-molecular cross-coupling reaction). In some cases, the metal-containing fragment and the electrophile fragment are present in different compounds (i.e., an inter-molecular cross-coupling reaction).

In some embodiments, the cross-coupling reactions disclosed herein for carbon-carbon bonds (i.e., carbon-carbon cross coupling reactions). In some embodiments, the carbon-carbon coupling reaction is a Kumada reaction, a Heck reaction, a Sonogashira reaction, a Negishi reaction, a Stille reaction, a Suzuki reaction, or a Fukuyama reaction. In some embodiments, the carbon-carbon coupling reaction is a Suzuki reaction (e.g., Suzuki-Miyaura). As is understood, Suzuki reactions typically comprise a cross-coupling reaction between a metal-containing compound (e.g., boron-containing compound) and an electrophile catalyzed by a palladium metal catalyst.

In some embodiments, the cross-coupling reaction is a reaction between a) a boron-containing compound, a zinc-containing compound, or a magnesium-containing compound and b) an electrophile. Examples of electrophiles are discussed below.

### Boron-Containing Compound

In some embodiments, the cross-coupling reaction is a reaction between a boron-containing compound and an electrophile. In some embodiments, the boron-containing compound is an aryl boronic acid, a heteroaryl boronic acid, an aryl boronic ester, or a heteroaryl boronic ester. In some embodiments, the boron-containing compound is a heteroaryl boronic acid.

The term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl, each of which may optionally be substituted with 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S—, nitro, cyano, carboxy, alkyl-O—C(O)—, carbamoyl, alkyl-S(O)—, sulfonyl, sulfonamido, phenyl, and heterocyclyl.

The term “heteroaryl” refers to ring having five to ten (5-10) members of which 1 to 4 (or 1 to 3) ring atoms are heteroatoms selected from N, O, and S, including monocyclic heteroaromatic rings and polycyclic aromatic rings in which a monocyclic aromatic ring is fused to one or more other aromatic ring.

Examples of heteroaryl groups include, but are not limited to, pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, benzotriazolyl, benzothiazolyl, benzoxazolyl, benzimidazolyl, isoquinolinyl, indolyl, isoindolyl, acridinyl, benzisoxazolyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

### Compound B

In some embodiments, the heteroaryl boronic acid is Compound B

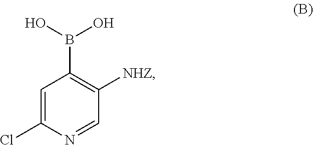

wherein Z is an amine protecting group. The amine protecting group, Z, can be any suitable amine protecting group. In some embodiments, Z is a carbamate, for example, tert-butoxycarbonyl (Boc) such that Compound B has a formula of

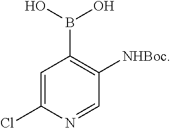 [SMILES: CC(C)(C)OC(=O)Nc1cnc(Cl)cc1B(O)O]

### Electrophiles

The electrophile can be any suitable electrophile capable of reacting with metal-containing compound in the cross-coupling reaction. In some embodiments, the electrophile is an alkenyl electrophile, an alkynyl electrophile, or an aryl electrophile. In some embodiments, the electrophile comprises a halide (e.g., F, Cl, Br, or I), sulfonate ester, or a sulfamate.

### Compound C

In some embodiments, in conjunction with other embodiments disclosed herein, the electrophile is Compound C

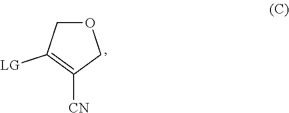

wherein LG is a leaving group. The leaving group LG can be any suitable leaving group. In some embodiments, LG is a sulfonate ester, a sulfamate, or a halide. In some embodiments, the sulfonate ester is tosyl, mesyl, nosyl, or triflyl. In some embodiments, the sulfonate ester is tosyl such that Compound C has a formula of

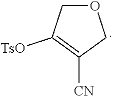 [SMILES: Cc1ccc(S(=O)(=O)OC2=C(C#N)COC2)cc1]

### Nickel(II) Precatalyst

As described herein, the disclosed cross-coupling reactions comprise using a nickel(II) precatalyst to produce a nickel(0) catalyst. The nickel(II) precatalyst can be any suitable precatalyst capable of producing a nickel(0) catalyst under the conditions described herein. In some embodiments, the nickel(II) precatalyst comprises a nickel(II) salt (e.g., nickel(II) halide). In some embodiments, the nickel(II) halide is NiCl₂dppp, NiCl₂DME, NiCl₂dppe, NiBr₂DME, NiCl₂·6H₂O, NiBr₂·3H₂O, NiI₂, NiCl₂, NiBr₂, NiCl₂(PPh₃)₂, NiCl₂(PCy₃)₂, or NiCl₂(dppf). In some embodiments, the nickel(II) precatalyst is NiCl₂DME.

The nickel(II) precatalyst is present in a suitable concentration. In some embodiments, the nickel(II) precatalyst is present at 0.01 to 20 mol %, based upon Compound B (e.g., 0.01, 0.05, 0.10, 0.50, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0. 16.5, 17.0, 17.5, 18.0, 18.5, 19.0, 19.5, or 20.0 mol % based upon Compound B). Alternatively, or in addition, in some embodiments the nickel(II) precatalyst can be present at 0.1 to 10 mol % based upon Compound B (e.g., 0.2 to 9.0, 0.3 to 8.0, 0.4 to 7.0, 0.5 to 6.0, 1.0 to 6.0, 2.0 to 6.0, or 3.0 to 6.0 mol %, based upon Compound B). In some embodiments, the nickel(II) precatalyst is present at 5.0 mol %, based upon Compound B.

### Electrolyte

As described herein, the disclosed cross-coupling reactions are conducted in an electrolyte in a solvent. The electrolyte can be any suitable electrolyte capable of conducting a current. Illustrative electrolytes include, for example, alkylammonium salts and/or imide salts or alkali metal salts. In some embodiments, the electrolyte is an alkylammonium salt, for example, alkali metal salts of NBu₄PF₆, NBu₄BF₄, NBu₄ClO₄, NBu₄Cl, NBu₄Br, or a combination thereof. In some embodiments, the electrolyte comprises a alkali metal salt of NBu₄PF₆.

In some embodiments, the alkali metal salt is LiCl, NaCl, KCl, KPF₆, LiClO₄, LiC₂F₆NO₄S₂, or a combination thereof.

In some embodiments, the electrolyte comprises LiC₂F₆NO₄S₂ (i.e., lithium bis(trifluromethanesulfonyl)imide).

The electrolyte is present in any suitable concentration. In some embodiments, the electrolyte is present at a concentration of 0.001 to 1.0 molar (e.g., 0.01, 0.05, 0.10, 0.10, 0.20, 0.30, 0.40, 0.50, 0.60, 0.70, 0.80, 0.90, or 1.0 molar). Alternatively, or in addition, in some embodiments the electrolyte can be present at a concentration of 0.01 to 0.5 molar (e.g., 0.01 to 0.4, 0.01 to 0.3, or 0.1 to 0.3 molar). In some embodiments, the electrolyte is present at a concentration of 0.1 molar. In some embodiments, the electrolyte is present at a concentration of 0.2 molar.

### Solvent

Any suitable solvent can be employed in the processes disclosed herein. In some embodiments, the solvent comprises acetonitrile, 2-methyltetrahydrofuran, tetrahydrofuran, dimethylacetamide, dimethylformamide, acetone, water, methanol, ethanol, isopropanol, toluene, dichloromethane (DCM), dimethylsulfoxide (DMSO), N-methyl-2-pyrrolidone (NMP), or a combination thereof. In some embodiments, the solvent comprises acetonitrile and 2-methyltetrahydrofuran.

The solvent is present in any suitable amount. Typically, the solvent is present in an amount of 0 to 50 L/kg (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 L/kg). Alternatively, or in addition, in some embodiments the solvent can be present in an amount of 1 to 25 L/kg (e.g., 1 to 20, 1 to 15, 2 to 10, or 2 to 5 L/kg). In some embodiments, the solvent is present in an amount of 4 L/kg.

### Phosphine

The disclosed cross-coupling reactions comprise producing a nickel(0) catalyst by exposing a mixture comprising a nickel(II) precatalyst, a phosphine, and an electrolyte to a current. Typically, the phosphine comprises a suitable ligand. In some embodiments, the phosphine comprises a monodentate phosphine ligand or a bidentate phosphine ligand.

In some embodiments, the monodentate phosphine ligand is P(nBu)₃, P(tBu)₃, or PCy₃.

In some embodiments, the bidentate phosphine ligand is XantPhos, dppe, dppp, or HP(nBu)₃-BF₄.

The phosphine is present in any suitable amount. In some embodiments, the phosphine is present at 0.03 to 50 mol %, based upon Compound B (e.g., 0.03, 0.05, 0.10, 0.50, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0. 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 41.5, 42.0, 42.5, 43.0, 43.5, 44.0, 44.5, 45.0, 45.5, 46.0, 46.5, 47.0, 47.5, 48.0, 49.5, or 50.0 mol %, based upon Compound B). Alternatively, or in addition, in some embodiments the phosphine can be present at 1.0 to 30 mol % based upon Compound B (e.g., 5.0 to 25.0, 5.0 to 20.0, or 10.0 to 20.0 mol %, based upon Compound B). In some embodiments, the phosphine is present at 15 mol %, based upon Compound B.

### Current

The disclosed cross-coupling reactions comprise producing a nickel(0) catalyst by exposing a mixture comprising a nickel(II) precatalyst, a phosphine, and an electrolyte to a current. Typically, the current is applied across an anode and a cathode. Illustrative anodes include, for example, anodes comprising graphite, SS, Ni, Zn, Al, Fe, Mg, glassy carbon, reticulated vitreous carbon, boron-doped diamond, platinum, copper, gold, or a combination thereof. In some embodiments, the anode comprises Zn, Mg, Al, Fe, Ni, or a combination thereof.

Illustrative cathodes include, for example, cathodes comprising graphite, SS, Ni, glassy carbon, Zn, Al, Fe, reticulated vitreous carbon, boron-doped diamond, platinum, copper, gold, or a combination thereof.

In some embodiments, in conjunction with other embodiments disclosed herein, the current has a density of 0 mA/cm² to −100 mA/cm² at the cathode and 0 mA/cm² to +100 mA/cm² at the anode. In some embodiments, in conjunction with other embodiments disclosed herein, the current has a density of −20 mA/cm² at the cathode and +20 mA/cm² at the anode.

In some embodiments, in conjunction with other embodiments disclosed herein, the charge (i.e., total charge passed) is about 2 to 10 F/mol (e.g., 2.0, 2.5, 3.0, 3.5, 4.0, 4.5, 5.0, 5.5, 6.0, 6.5, 7.0, 7.5, 8.0, 8.5, 9.0, 9.5, or 10 F/mol). In some embodiments, the total charge passed is 2-10 F/mol (e.g., 3-9 F/mol, 4-8 F/mol, or 5-7 F/mol). In some embodiments, the total charge passed is 5 F/mol.

### Base

In some embodiments, the mixture comprising the nickel(II) precatalyst and electrolyte further comprises a base. Illustrative suitable bases include, for example, an alkylamine base. In some embodiments, the alkylamine base is triethylamine, diisopropylethyamine, N-methylmorpholine, N-methylpiperidine, or a combination thereof. In some embodiments, the alkylamine base comprises trimethylamine.

The base is present in a suitable amount. In some embodiments, the base is present in 0.1 to 5.0 equivalents, based upon Compound B (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 equivalents, based upon Compound B). Alternatively, or in addition, in some embodiments the base can be present in 0.1 to 2.0 equivalents, based upon Compound B (e.g., 0.1 to 1.5, 0.1 to 1.0, 0.1 to 0.9, 0.1 to 0.8, 0.1 to 0.7, 0.1 to 0.6, or 0.2 to 0.5 equivalents, based upon Compound B). In some embodiments, the base is present in 0.3 equivalents, based upon Compound B.

In some embodiments, the mixture comprising the nickel(II) precatalyst and electrolyte does not comprise a base. In these embodiments, the anode may be referred to as a “sacrificial anode” and comprises Zn, Mg, Al, Fe, Ni, or a combination thereof.

### Cross-Coupling Reaction Temperature

The disclosed cross-coupling reactions are conducted at a suitable temperature. Typically, the cross-coupling reactions are conducted at a temperature of 20 to 80° C. (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C. Alternatively, or in addition, in some embodiments the reactions can be conducted at a temperature of, for example, 25 to 75° C., 30 to 70° C., 35 to 65° C., or 40 to 60° C. In some embodiments, the cross-coupling reaction is conducted at a temperature of 70° C.

### Processes for Compound A

In some embodiments, the disclosure provides processes for preparing Compound A or a salt thereof comprising using the cross-coupling processes disclosed herein. In some embodiments, in conjunction with other embodiments disclosed herein, the disclosure provides a process for preparing Compound A or a salt thereof comprising (a) preparing a nickel(0) catalyst by exposing a mixture comprising a nickel(II) precatalyst, a phosphine, and an electrolyte in a solvent to a current to produce the nickel(0) catalyst; and (b) contacting the nickel(0) catalyst with Compound B and Compound C in an admixing solvent to form Compound A or a salt thereof, as described herein.

In some embodiments, the disclosure provides processes for preparing Compound A or a salt thereof comprising (a) preparing a nickel(0) catalyst by exposing a mixture comprising a nickel(II) precatalyst (e.g., NiCl₂DME), a phosphine (e.g. HP(n-Bu)₃BF₄), and an electrolyte (NBu₄PF₆ or LiC₂F₆NO₄S₂) in a solvent (e.g., acetonitrile or 2-MeTHF) to a current to produce the nickel(0) catalyst; and (b) contacting the nickel(0) catalyst with Compound B of formula

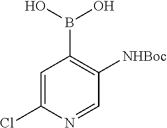 [SMILES: CC(C)(C)OC(=O)Nc1cnc(Cl)cc1B(O)O]

and Compound C of formula of

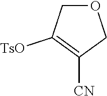 [SMILES: Cc1ccc(S(=O)(=O)OC2=C(C#N)COC2)cc1]

in an admixing solvent (e.g., 2-MeTHF and water) to form Compound A or a salt thereof.

### Admixing Solvent

In some embodiments, the admixing solvent comprises acetonitrile, 2-methyltetrahydrofuran, dimethylacetamide, dimethylformamide, acetone, water, methanol, ethanol, isopropanol, toluene, DCM, DMSO, NMP, water, or a combination thereof. In some embodiments, the admixing solvent is a mixture of an organic solvent and water. In some embodiments, the admixing solvent comprises 2-methyltetrahydrofuran and water.

The admixing solvent is present in a suitable amount. Typically, the admixing solvent is present in 1.0 to 50 L/kg (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 L/kg). Alternatively, or in addition, in some embodiments the solvent can be present in an amount of 1 to 25 L/kg (e.g., 1 to 20, 1 to 15, 2 to 10, or 2 to 5 L/kg). In some embodiments, the admixing solvent is present in 22 L/kg, for example, 4 L/kg of water and 18 L/kg of 2-methyltetrahydrofuran.

In some embodiments, the admixing solvent further comprises an inorganic salt. In some embodiments, the inorganic salt comprises potassium phosphate (K₃PO₄).

The inorganic salt is present in a suitable amount. Typically, the inorganic salt is present in 1 to 5 equivalents (e.g., 1.0, 1.1, 1.2, 1.3, 1.4 1.5, 1.6, 1.7, 1.8. 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 equivalents). In some embodiments, the inorganic salt is present in 2 to 3 equivalents (e.g., 2.5 equivalents).

### Processes for Compound F

In some embodiments, the disclosure provides processes for preparing Compound F

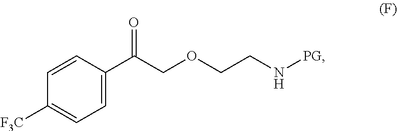

wherein PG is a protecting group. The PG protecting group can be any suitable protecting group. In some embodiments, PG is a carbamate, a trialkylsilyl, a benzyl, a trityl or a sulfonyl protecting group. Exemplary carbamates include Boc, FMOC, and CZB (benzyloxy carbonyl).

Exemplary trialkylsilyls include TBS, TMD, TBDPS, and TES. In some embodiments, PG is Boc, FMOC, or BnO.

In some embodiments, PG is Boc.

As described herein, the disclosed processes for preparing Compound F provide several advantages over conventional processes (e.g., Grignard processes), including faster reaction times and milder conditions.

The disclosure provides processes for preparing Compound F comprising (a) reacting Compound H

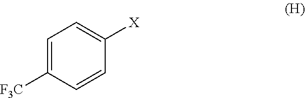

in a first solvent with an organolithium reagent to form a lithiated intermediate; and (b) reacting the lithiated intermediate with Compound G

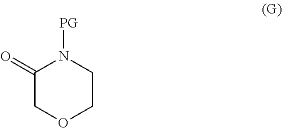

in a second solvent to form Compound F, wherein X is halogen (e.g., F, Cl, Br, or I). In some embodiments, X is Br.

### Organolithium Reagent

In some embodiments, the organolithium reagent is an alkylithium reagent. In some embodiments, the organolithium reagent is nBuLi.

The organolithium reagent is present in a suitable amount. In some embodiments, the organolithium reagent is present at 1.1 to 3 molar equivalents based upon on Compound H. Accordingly, the organolithium reagent can be present at 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 molar equivalents based upon on Compound H. In some embodiments, the organolithium reagent is present at 1.0 to 3.0, 1.5 to 3.0, 1.5 to 2.5, or 2.0 to 3.0 molar equivalents based upon on Compound H. In some embodiments, the organolithium reagent is present at 2.5 molar equivalents based upon on Compound H. In some embodiments, the organolithium reagent is present at 1.1 molar equivalents based upon on Compound H.

### First and Second Solvents

The processes for preparing Compound F are conducted in a first solvent and a second solvent. In some embodiments, the first solvent comprises 2-methyltetrahydrofuran, tetrahydrofuran, 1,2-dimethoxyethane, 1,4-dioxane, tert-butyl methyl ether, cyclopentyl methyl ether, toluene, heptane, hexane, cyclohexane, or a combination thereof. In some embodiments, the first solvent comprises 2-methyltetrahydrofuran.

In some embodiments, the second solvent comprises 2-methyltetrahydrofuran, tetrahydrofuran, 1,2-dimethoxyethane, 1,4-dioxane, tert-butyl methyl ether, cyclopentyl methyl ether, toluene, or a combination thereof. In some embodiments, the second solvent comprises 2-methyltetrahydrofuran.

In some embodiments, the first solvent and the second solvent are the same.

### Temperature

The processes disclosed herein for preparing Compound F are conducted at a suitable temperature.

In some embodiments, the process is conducted at a temperature of −70° C. to −50° C. (e.g., −65 to −55° C. and −60° C.). In some embodiments, the reaction is conducted at a temperature of −60° C.

### Acidic Solution

In some embodiments, the disclosed processes further comprise (c) admixing Compound F and an acidic solution to form a quenched reaction mixture.

The acidic solution, when present, can comprise any suitable acid. For example, In some embodiments, the acidic solution comprises hydrochloric acid. In some embodiments, the acidic solution further comprises an organic solvent. For example, In some embodiments, the acidic solution comprises hydrochloric acid and isopropanol.

In embodiments comprising an acidic solution, the acidic solution has a suitable acid concentration. For example, In some embodiments, the acidic solution has an acid concentration of 0.5 to 4 molar (e.g., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 molar). In some embodiments, the acidic solution has a concentration of 0.5 to 3.5 molar, 0.5 to 3.0 molar, 0.5 to 2.5 molar, 0.5 to 2.0 molar, 0.5 to 1.5 molar, or 1.0 to 1.5 molar. In some embodiments, the acidic solution has a concentration of 1.3 molar.

In some embodiments, the acidic solution is present at 2.5 to 8 volume equivalents based upon Compound G. For example, the acidic solution can be present at 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0 volume equivalents based upon Compound G. In some embodiments, the acidic solution can be present at 2.5 to 8, 3.0 to 7.5, 3.5 to 7.0, 4.0 to 6.5, or 4.5 to 6.0 volume equivalents based upon Compound G.

### Pre-Crystallization Mixture

In some embodiments, the disclosed processes further comprises mixing the quenched reaction mixture with aqueous citric acid, brine, or both to form a pre-crystallization mixture. In some embodiments, the disclosed processes further comprise crystallizing Compound F from the pre-crystallization mixture to provide crystalline Compound F.

In embodiments comprising crystallizing Compound F, the crystallizing is conducted using toluene and n-heptane as crystallizing solvents.

### Continuous Process

In some embodiments, the processes for preparing Compound F are conducted in a continuous process apparatus or in semi-batch mode, as described herein. When conducted as a continuous process, the continuous process (or process step) is conducted in a suitable apparatus (e.g., plug-flow reactor).

Moreover, the continuous processes disclosed herein are conducted at a suitable flow rates to provide suitable residence times. Typically, the processes are conducted at a total flow rate of 2 to 5,000 mL/min (e.g, 5, 25, 50, 75, 100, 150, 200, 250, 300, 350, 400, 450, 500, 550, 600, 650, 700, 750, 800, 850, 900, 950, 1,000, 1,250, 1,500, 1,750, 2,000, 2,250, 2,500, 2,750, 3,000, 3,250, 3,500, 3,750, 4,000, 4,250, 4,500, 4,750, or 5,000 mL/min). For example, In some embodiments, the disclosed processes are conducted at total flow rate of 60 mL/min. Alternatively, or in addition, the flow rate of each step of the process is conducted at a suitable flow rate. In some embodiments, each step of the process is conducted at a flow rate that corresponds to the total flow rate described herein. In some embodiments, each step of the processes are conducted at flow rate of 15 to 100 mL/min (e.g., 15, 16, 17, 18, 19, 20, 21, 22, 23, 24, 25, 30, 35, 40, 45, 50, 55, 60, 65, 70, 75, 80, 85, 90, 95, or 100 mL/min). In some embodiments, in conjunction with other embodiments disclosed herein, the processes are conducted to have a total residence time of 10 seconds to 5 minutes (e.g., 10 s, 15 s, 30 s, 45 s, 60 s, 1 min, 1.5 min, 2 min, 2.5 min, 3.0 min, 3.5 min, 4.0 min, 4.5 min, or 5 min). In some embodiments, the residence time is 5 s to 1 min. (e.g., 15 s to 1 min)

### Processes for Compound I

In various embodiments, in conjunction with other embodiments disclosed herein, the disclosure provides processes for preparing Compound I

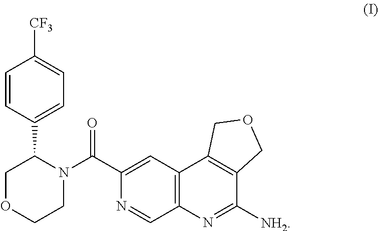 [SMILES: Nc1nc2cnc(C(=O)N3CCOC[C@@H]3c3ccc(C(F)(F)F)cc3)cc2c2c1COC2]

For example, Compound F prepared according to the processes disclosed herein can be used in subsequent processes to provide Compound I. For example, Compound F prepared according to the processes disclosed herein can be converted to a compound of formula

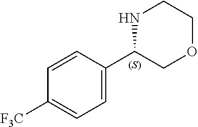

according to the Scheme below.

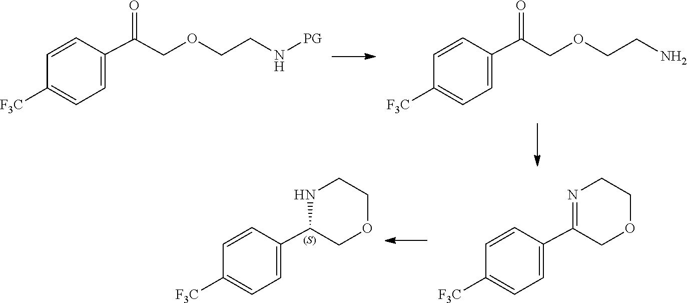

A compound of formula

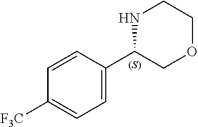

can be reacted with Compound A or a salt thereof to produce Compound (I), wherein Compound A or a salt thereof can be prepared according to the processes herein. This is shown in the retrosynthetic analysis of Compound (I) shown below.

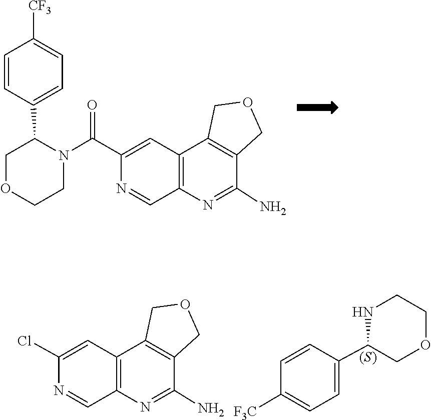

### EMBODIMENTS

1. A process for a cross-coupling reaction comprising (a) exposing a mixture comprising a nickel(II) precatalyst, a phosphine, and an electrolyte in a solvent to a current to produce a nickel(0) catalyst; and (b) catalyzing the cross-coupling reaction with the nickel(0) catalyst.
2. The process of embodiment 1, wherein the cross-coupling reaction is a carbon-carbon cross coupling reaction.
3. The process of embodiment 1 or 2, wherein the carbon-carbon coupling reaction is a Kumada reaction, a Heck reaction, a Sonogashira reaction, a Negishi reaction, a Stille reaction, a Suzuki reaction, or a Fukuyama reaction.
4. The process of embodiment 3, wherein the carbon-carbon coupling reaction is a Suzuki-Miyaura reaction.
5. The process of any one of embodiments 1-4, wherein the cross-coupling reaction is a reaction between a) a boron-containing compound, a zinc-containing compound, or a magnesium-containing compound and b) an electrophile.
6. The process of embodiment 5, wherein the boron-containing compound is an aryl boronic acid, a heteroaryl boronic acid, an aryl boronic ester, or a heteroaryl boronic ester.
7. The process of embodiment 6, wherein the boron-containing compound is a heteroaryl boronic acid.
8. The process of any one of embodiments 5-7, wherein the electrophile is an alkenyl electrophile, an alkynyl electrophile, or an aryl electrophile.
9. The process of any one of embodiments 5-8, wherein the electrophile comprises a halide, sulfonate ester, or a sulfamate.
10. The process of any one of embodiments 1-9, wherein the nickel(II) precatalyst comprises a nickel(II) salt.
11. The process of embodiment 10, wherein the nickel(II) salt comprises a nickel(II)halide.
12. The process of embodiment 11, wherein the nickel(II)halide is NiCl₂dppp, NiCl₂DME, NiCl₂dppe, NiBr₂DME, NiCl₂-6H₂O, NiBr₂-3H₂O, NiI₂, NiCl₂, NiBr₂, NiCl₂(PPh₃)₂, NiCl₂(PCy₃)₂, or NiCl₂(dppf).
13. The process of any one of embodiments 10-12, wherein the nickel(II) precatalyst is NiCl₂DME.
14. The process of any one of embodiments 1-13, wherein the electrolyte comprises an alkylammonium salt, an imide salt, or a combination thereof.
15. The process of embodiment 14, wherein the alkylammonium salt is an alkali metal salt of NBu₄PF₆, NBu₄BF₄, NBu₄ClO₄, NBu₄Cl, NBu₄Br, or a combination thereof.
16. The process of embodiment 15, wherein the alkali metal salt is LiCl, NaCl, KCl, KPF₆, LiClO₄, or a combination thereof.
17. The process of any one of embodiments 1-16, wherein the electrolyte comprises an alkali metal salt of NBu₄PF₆.
18. The process of any one of embodiments 1-17, wherein the electrolyte comprises LiC₂F₆NO₄S₂.
19. The process of any one of embodiments 1-18, wherein the solvent comprises acetonitrile, 2-methyltetrahydrofuran, tetrahydrofuran, dimethylacetamide, dimethylformamide, acetone, water, methanol, ethanol, isopropanol, toluene, dichloromethane, dimethylsulfoxide, N-methyl-2-pyrrolidone, or a combination thereof.
20. The process of embodiment 19, wherein the solvent comprises acetonitrile and 2-methyltetrahydrofuran.
21. The process of any one of embodiments 1-20, wherein the phosphine comprises a monodentate phosphine ligand or a bidentate phosphine ligand.
22. The process of embodiment 21, wherein the monodentate phosphine ligand is P(nBu)₃, P(tBu)₃, or PCy₃.
23. The process of embodiment 21, wherein the bidentate phosphine ligand is XantPhos, dppe, dppp), or HP(nBu)₃-BF₄.
24. The process of embodiment 23, wherein the phosphine comprises HP(nBu)₃-BF₄.
25. The process of any one of embodiments 1-24, wherein the current is applied across an anode and a cathode.
26. The process of embodiment 25, wherein the anode comprises graphite, SS, Ni, Zn, Al, Fe, Mg, glassy carbon, reticulated vitreous carbon, boron-doped diamond, platinum, copper, gold, or a combination thereof.
27. The process of embodiment 26, wherein the anode comprises Zn, Mg, Al, Fe, Ni, or a combination thereof.
28. The process of any one of embodiments 25-27, wherein the cathode comprises graphite, SS, Ni, glassy carbon, Zn, Al, Fe, reticulated vitreous carbon, boron-doped diamond, platinum, copper, gold, or a combination thereof.
29. The process of any one of embodiments 1-28, wherein the current has a density of 0 mA/cm² to −100 mA/cm² at the cathode and 0 mA/cm² to +100 mA/cm² at the anode.
30. The process of embodiment 29, wherein the current has a density of −20 mA/cm² at the cathode and +20 mA/cm² at the anode.
31. The process of any one of embodiments 1-26 and 28-30, wherein the mixture further comprises a base.
32. The process of embodiment 31, wherein the base comprises an alkylamine base.
33. The process of embodiment 32, where the alkylamine base is triethylamine, diisopropylethyamine, N-methylmorpholine, N-methylpiperidine, or a combination thereof.
34. The process of embodiment 33, wherein the alkylamine base comprises triethylamine.
35. A process for preparing Compound A, or a salt thereof:

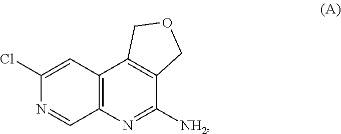 [SMILES: Nc1nc2cnc(Cl)cc2c2c1COC2]

comprising
(a) preparing a nickel(0) catalyst by exposing a mixture comprising a nickel(II) precatalyst, a phosphine, and an electrolyte in a solvent to a current to produce the nickel(0) catalyst; and
(b) contacting the nickel(0) catalyst with Compound B and Compound C in an admixing solvent to form Compound A or a salt thereof:

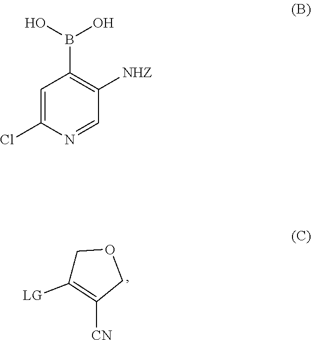

wherein Z is an amine protecting group and LG is a leaving group.
36. The process of embodiment 35, wherein the nickel(II) precatalyst is a nickel(II) salt.
37. The process of embodiment 36, wherein the nickel(II) salt is a nickel(II)halide.
38. The process of embodiment 37, wherein the nickel(II)halide is NiCl₂dppp, NiCl₂DME, NiCl₂dppe, NiBr₂DME, NiCl₂-6H₂O, NiBr₂-3H₂O, NiI₂, NiCl₂, NiBr₂, NiCl₂(PPh₃)₂, NiCl₂(PCy₃)₂, or NiCl₂(dppf).
39. The process of any one of embodiments 35-38, wherein the nickel(II) precatalyst is NiCl₂DME.
40. The process of embodiment 35-39, wherein the electrolyte comprises an alkylammonium salt, an imide salt, or a combination thereof.
41. The process of embodiment 40, wherein the alkylammonium salt is an alkali metal salt of NBu₄PF₆, NBu₄BF₄, NBu₄ClO₄, NBu₄Cl, NBu₄Br, or a combination thereof.
42. The process of embodiment 41, wherein the alkali metal salt is LiCl, NaCl, KCl, KPF₆, LiClO₄ or a combination thereof.
43. The process of any one of embodiments 35-42, wherein the electrolyte comprises NBu₄PF₆, LiC₂F₆NO₄S₂, or a combination thereof.
44. The process of embodiment 43, wherein the electrolyte comprises an alkali metal salt of NBu₄PF₆.
45. The process of embodiment 44, wherein the electrolyte comprises LiC₂F₆NO₄S₂.
46. The process of any one of embodiments 35-45, wherein solvent comprises acetonitrile, 2-methyltetrahydrofuran, tetrahydrofuran, dimethylacetamide, dimethylformamide, acetone, water, methanol, ethanol, isopropanol, toluene, dichloromethane dimethylsulfoxide, N-methyl-2-pyrrolidone, or a combination thereof.
47. The process of embodiment 46, wherein the solvent comprises acetonitrile and 2-methyltetrahydrofuran.
48. The process of any one of embodiments 35-47, wherein the phosphine comprises a monodentate phosphine ligand or a bidentate phosphine ligand.
49. The process of embodiment 48, wherein the monodentate phosphine ligand is P(nBu)₃, P(tBu)h, or PCy₃.
50. The process of embodiment 48, wherein the bidentate phosphine ligand is XantPhos, dppe, dppp), or HP(nBu)₃-BF₄.
51. The process of embodiment 50, wherein the phosphine comprises HP(nBu)₃-BF₄.
52. The process of any one of embodiments 35-51, wherein the current is applied across an anode and a cathode.
53. The process of embodiment 52, wherein the anode comprises graphite, SS, Ni, Zn, Al, Fe, Mg, glassy carbon, reticulated vitreous carbon, boron-doped diamond, platinum, copper, gold, or a combination thereof.
54. The process of embodiment 53, wherein the anode comprises Zn, Mg, Al, Fe, Ni, or a combination thereof.
55. The process of any one of embodiments 52-54, wherein the cathode comprises graphite, SS, Ni, graphite, SS, Ni, Zn, Al, Fe, Mg, glassy carbon, reticulated vitreous carbon, boron-doped diamond, platinum, copper, gold, or a combination thereof.
56. The process of any one of embodiments 35-55, wherein the current has a density of 0 mA/cm² to −100 mA/cm² at the cathode and 0 mA/cm² to +100 mA/cm² at the anode.
57. The process of embodiment 56, wherein the current has a density of −20 mA/cm² at the cathode and +20 mA/cm² at the anode (5 F/mol).
58. The process of any one of embodiments 35-53 and 55-57, wherein the mixture further comprises a base.
59. The process of embodiment 58, wherein the base comprises an alkylamine base
60. The process of embodiment 59, wherein the alkylamine base comprises triethylamine, diisopropylethyamine, N-methylmorpholine, N-methylpiperidine), or a combination thereof.
61. The process of embodiment 59 or 60, wherein the base comprises triethylamine.
62. The process of any one of embodiments 35-61, wherein Z is a carbamate.
63. The process of embodiment 63, wherein the carbamate is tert-butoxycarbonyl (Boc).
64. The process of any one of embodiments 35-63, wherein LG is a sulfonate ester, a sulfamate, or a halide.
65. The process of embodiment 64, wherein the sulfonate ester is tosyl, mesyl, nosyl, or triflyl.
66. The process of embodiment 66, wherein the sulfonate ester is tosyl.
67. The process of any one of embodiments 35-66, wherein the admixing solvent comprises acetonitrile, 2-methyltetrahydrofuran, dimethylacetamide, dimethylformamide, acetone, water, methanol, ethanol, isopropanol, toluene, DCM, DMSO, NMP, water, or a combination thereof.
68. The process of embodiment 67, wherein the admixing solvent comprises 2-methyltetrahydrofuran and water.
69. The process of any one of embodiments 35-68, wherein the admixing solvent further comprises an inorganic salt.
70. The process of embodiment 69, wherein the inorganic salt comprises potassium phosphate (K₃PO₄).
71. The process of any one of embodiments 35-70, wherein the admixing is conducted in continuous mode.
72. A process for preparing Compound F

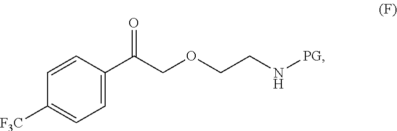

comprising
(a) reacting Compound H

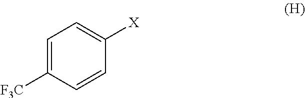

in a first solvent with an organolithium reagent to form a lithiated intermediate; and
(b) reacting the lithiated intermediate with Compound G

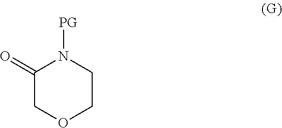

in a second solvent to form Compound F,
wherein X is halogen and PG is a protecting group.
73. The process of embodiment 72, wherein the organolithium reagent is an alkyllithium reagent.
74. The process of embodiment 73, wherein the organolithium reagent is nBuLi.
75. The process of any one of embodiments 72-74, wherein the organolithium reagent is present at 1.1 to 3 molar equivalents based upon Compound H.
76. The process of embodiment 75, wherein the organolithium reagent is present at 1.1 molar equivalents based upon Compound H.
77. The process of any one of embodiments 72-76, wherein the first solvent comprises 2-methyltetrahydrofuran, tetrahydrofuran, 1,2-dimethoxyethane, 1,4-dioxane, tert-butyl methyl ether, cyclopentyl methyl ether, toluene, heptane, hexane, cyclohexane, or a combination thereof.
78. The process of embodiment 77, wherein the first solvent comprises 2-methyltetrahydrofuran.
79. The process of any one of embodiments 72-78, wherein the second solvent comprises 2-methyltetrahydrofuran, tetrahydrofuran, 1,2-dimethoxyethane, 1,4-dioxane, tert-butyl methyl ether, cyclopentyl methyl ether, toluene, or a combination thereof.
80. The process of embodiment 79, wherein the second solvent comprises 2-methyltetrahydrofuran.
81. The process of any one of embodiments 72-80, wherein the first and second solvents are the same.
82. The process of any one of embodiments 72-81, wherein X is F, Cl, Br, or I.
83. The process of embodiment 82, wherein X is Br.
84. The process of any one of embodiments 72-83, wherein PG is a carbamate, a trialkylsilyl, a benzyl, a trityl or a sulfonyl protecting group.
85. The process of embodiment 84, wherein PG is Boc, FMOC, or BnO.
86. The process of embodiment 85, wherein PG is Boc.
87. The process of any one of embodiments 72-86, wherein the process is conducted at a temperature of −70 to −50° C.
88. The process of any one of embodiments 71-87, further comprising (c) admixing Compound F and an acidic solution to form a quenched reaction mixture.
89. The process of embodiment 88, wherein the acidic solution comprises hydrochloric acid.
90. The process of embodiment 88 or 89, wherein the acidic solution comprises hydrochloric acid and isopropanol.
91. The process of any one of embodiments 88-90, wherein the acidic solution has an acid concentration of 0.5 to 4 molar.
92. The process of embodiment 91, wherein the acidic solution has a concentration of 1.3 molar.
93. The process of any one of embodiments 88-92, wherein the acidic solution is present at 2.5 to 8 volume equivalents based upon Compound G.
94. The process of any one of embodiments 99-93, further comprising mixing the quenched reaction mixture with aqueous citric acid, brine, or both to form a pre-crystallization mixture.
95. The process of embodiment 94, further comprising crystallizing Compound F from the pre-crystallization mixture to provide crystalline Compound F.
96. The process of embodiment 95, wherein the crystallizing is conducted using toluene and n-heptane as crystallizing solvents.
97. The process of any one of embodiments 72-96, conducted in a continuous process apparatus or in semi-batch mode.
98. The process of embodiment 97, wherein the continuous process apparatus is a plug-flow reactor.
99. The process of embodiment 97 or 98, wherein steps a, b, and optionally c have a total residence time of 10 seconds to 5 minutes.
100. The process of embodiment 99, wherein the residence time is 5 seconds to 1 minute.
101. The process of any one of embodiments 98-100, wherein steps a, b, and optionally c have a flow rate of 15 to 100 mL/min.
102. The process of embodiment 101, having a total flow rate of 60 mL/min.
103. The process of any one of embodiments 72-102, further comprising using Compound F to prepare Compound I

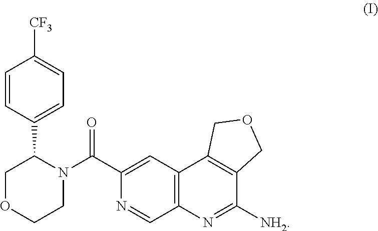 [SMILES: Nc1nc2cnc(C(=O)N3CCOC[C@@H]3c3ccc(C(F)(F)F)cc3)cc2c2c1COC2]

### EXAMPLES

The following examples further illustrate the disclosed processes, but of course, should not be construed as in any way limiting their scope.

### Example 1

All charges with respect to aryl boronic acid (i.e., Compound B).

### Nickel(0) Catalyst Preparation

A first reaction vessel was charged with NiCl₂DME (5 mol %) as a nickel(II) precatalyst, a tetrabutylammonium tetrafluoroborate (15 mol %) as a phosphine, supporting electrolyte NBu₄PF₆ (0.1 M), and acetonitrile (4 L/kg) as a solvent. The reactor was equipped with a graphite cathode and graphite anode and sealed. The reaction vessel was sparged with nitrogen and triethylamine (0.3 equiv) was added as a base. While under nitrogen, the reaction was agitated and current density at the cathode was maintained at −20 mA/cm² until the required charge was passed (5 F/mol).

### Cross-Coupling

A reaction vessel was charged with the Compound B, Z=Boc (1 equiv.) and 2-MeTHF (9 L/kg). The reaction vessel was sparged with nitrogen. A second reaction vessel was charged with the Compound C, LG=OTs (1.5 equiv.) and 2-MeTHF (9 L/kg) as a solvent. The second reaction vessel was sparged with nitrogen, charged with water, and sparged with nitrogen. A third reaction vessel was charged with potassium phosphate (2.5 equiv.). Water (4 L/kg) from the second reaction vessel was charged to the third reaction vessel and agitated until all the potassium phosphate was dissolved. Contents of the third reaction vessel were charged to the second reaction vessel. The second reaction vessel was agitated and heated to 70° C. The prepared nickel catalyst solution from the first reaction vessel was charged to the second reaction vessel. The contents of the first reaction vessel 1 were charged to reaction vessel 2 over 2.5 h and agitated at 70° C. for 4 h. The second reaction vessel was then cooled to 20° C. and held for 12 h.

### Workup and Crystallization

The second reaction vessel was charged with HCl (2M, 6 L/kg), to provide a crystalline solid, which was then filtered. The resulting cake was washed with water (10 L/kg) and IPA (10 L/kg) to yield Compound A (75% yield).

### Example 2

### Compound (A)—Electrochemical Catalyst Preparation Procedure:

All charges are with respect to DABOchlor.

### DABOchlor Deprotection

Reactor 1 was charged with DABOchlor (1 equiv.) of formula

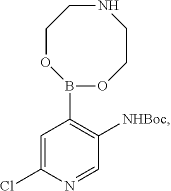 [SMILES: CC(C)(C)OC(=O)Nc1cnc(Cl)cc1B1OCCNCCO1]

2-MeTHF (9 L/kg), water (8.8 L/kg), and acetic acid (0.23 L/kg). The contents of reactor 1 were then agitated for 1 hour at 20° C. A phase split was performed and the aqueous layer was removed. In vessel 1, a solution of 10 wt % NaCl in water (9 L/kg) was prepared. The contents of vessel 1 were then charged to reactor 1. Reactor 1 was agitated for 1 hour. A phase split was performed and the aqueous layer was removed. The organic layer was retained for use in later steps.

### Catalyst Preparation

Reactor 2 was charged with NiCl₂DME (5 mol %), tributylphosphonium tetrafluoroborate (15 mol %), supporting electrolyte (either 0.2 M LiC₂F₆NO₄S₂ or 0.1 M NBu₄PF₆), and solvent (4 L/kg, either MeCN or 2-MeTHF). Reactor 2 was equipped with a cathode (graphite, Ni, SS) and an anode (graphite, Zn, Ni) and sealed. Reactor 2 was sparged with nitrogen. Triethylamine (0.3 equiv) was charged to reactor 2. While under nitrogen, the reaction was agitated and current density at the cathode was maintained at −20 mA/cm² until the required charge was passed (5 F/mol).

### Suzuki Cross-Coupling

Reactor 3 was charged with Compound C (1.5 equiv.) and 2-MeTHF (9 L/kg). Reactor 3 was sparged with nitrogen. Vessel 2 was charged with water and sparged with nitrogen. Vessel 3 was charged with K₃PO₄ (2.5 equiv.). Water (4 V) from vessel 2 was charged to vessel 3 and agitated until all K₃PO₄ was dissolved. Contents of vessel 3 were charged to reactor 3. Reactor 3 was agitated and heated to 70° C. The prepared nickel catalyst solution from reactor 2 was charged to reactor 3. The contents of reactor 1 were charged to reactor 3 over 2.5 h. Reactor 3 was agitated at 70° C. for 4 h. Reactor 3 was then cooled to 20° C. and held for 12 h.

### Workup and Crystallization

Reactor 3 was charged with HCl (2 M, 6 L/kg). The contents of reactor 3 were then filtered. The resulting cake was washed with water (10 L/kg) and IPA (10 L/kg) to provide the hydrochloride salt of Compound A.

### Example 3

### Flow Lithiation Procedure:

Compound H, X-Br (1 M solution in 2-MeTHF, 1.05 equiv.) was lithiated with n-hexyllithium (2.3 M in hexanes, 1.12 equiv.) in a pre-cooled −60° C. plug flow reactor (11 second residence time). This stream was added to (Compound G, PG=Boc) (1 M in 2-MeTHF, 1.0 equiv.) and was reacted in a second pre-cooled −60° C. plug flow reactor (7 second residence time). The addition stream was quenched with HCl (1.25 M in IPA, 1.05 equiv.) in a third pre-cooled −60° C. plug flow reactor (5 second residence time). The product stream was quenched with 8.7 L/kg 2.5 wt % aq. citric acid. After the flow process was complete, the product stream was charged to a reactor and the solution was mixed for 10 minutes. The lower aqueous phase was removed, and the organic phase was washed with 10.4 L/kg of 0.1 M potassium phosphate buffer (pH 7.4), followed by 8.7 L/kg of deionized water. The crude organic phase was heated to 50° C. (jacket temperature) and was distilled until approximately 4.3 L/kg remained. The solvent was then exchanged to toluene through two put-and-take distillations. On the last distillation the solution was concentrated until approximately 5.2 L/kg remained. The solution was then heated to 45° C., and 8.7 L/kg of heptane was dosed over 30 minutes. The solution was seeded with 0.5 wt % of pure Compound F, PG=Boc and was subsequently cooled to 0° C. over 2 hours. After holding for 2 hours at 0° C., the crystallized material was filtered, and the reactor and cake were washed with 5.2 L/kg of 2:1 heptane:toluene (pre-cooled to 0° C.), followed by 5.2 L/kg of heptane. The cake was dried under nitrogen stream for 12 hours, resulting in Compound F, PG=Boc (74.8% yield).

The lithiation can also be performed with n-butyllithium (2.5 M in hexanes or 1.6 M in hexanes) without any significant change in purity. During development, batch reactions could also be quenched with 3 M HCl in cyclopentylmethyl ether (CPME) without significant change in LC purity.

All references, including publications, patent applications, and patents, cited herein are hereby incorporated by reference to the same extent as if each reference were individually and specifically indicated to be incorporated by reference and were set forth in its entirety herein.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

The use of the terms “a” and “an” and “the” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (for example, “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed:

1. A process for a cross-coupling reaction comprising (a) exposing a mixture comprising a nickel(II) precatalyst, a phosphine, and an electrolyte in a solvent to a current to produce a nickel(0) catalyst; and (b) catalyzing the cross-coupling reaction with the nickel(0) catalyst.

2. The process of claim 1, wherein the cross-coupling reaction is a reaction between a) a boron-containing compound, a zinc-containing compound, or a magnesium-containing compound and b) an electrophile.

3. The process of claim 2, wherein the boron-containing compound is an aryl boronic acid, a heteroaryl boronic acid, an aryl boronic ester, or a heteroaryl boronic ester.

4. The process of claim 2 or 3, wherein the electrophile comprises a halide, sulfonate ester, or a sulfamate.

5. The process of any one of claims 1-4, wherein the nickel(II) precatalyst comprises a nickel(II) salt.

6. The process of claim 5, wherein the nickel(II) precatalyst is NiCl₂DME.

7. The process of any one of claims 1-6, wherein the electrolyte comprises an alkylammonium salt, an imide salt, or a combination thereof.

8. The process of claim 7, wherein the electrolyte comprises an alkali metal salt of NBu₄PF₆.

9. The process of claim 7 or 8, wherein the electrolyte comprises LiC₂F₆NO₄S₂.

10. The process of any one of claims 1-9, wherein the phosphine comprises a monodentate phosphine ligand or a bidentate phosphine ligand.

11. The process of claim 10, wherein the monodentate phosphine ligand is P(nBu)₃, P(tBu)₃, or PCy₃.

12. The process of claim 10, wherein the bidentate phosphine ligand is XantPhos, dppe, dppp), or HP(nBu)₃-BF₄.

13. The process of claim 12, wherein the phosphine comprises HP(nBu)₃-BF₄.

14. The process of any one of claims 1-13, wherein the current is applied across an anode and a cathode.

15. The process of claim 14, wherein the anode comprises graphite, SS, Ni, Zn, Al, Fe, Mg, glassy carbon, reticulated vitreous carbon, boron-doped diamond, platinum, copper, gold, or a combination thereof.

16. The process of claim 15, wherein the anode comprises Zn, Mg, Al, Fe, Ni, or a combination thereof.

17. The process of any one of claims 14-16, wherein the cathode comprises graphite, SS, Ni, glassy carbon, Zn, Al, Fe, reticulated vitreous carbon, boron-doped diamond, platinum, copper, gold, or a combination thereof.

18. The process of any one of claims 1-17, wherein the current has a density of 0 mA/cm² to −100 mA/cm² at the cathode and 0 mA/cm² to +100 mA/cm² at the anode.

19. The process of claim 18, wherein the current has a density of −20 mA/cm² at the cathode and +20 mA/cm² at the anode.

20. The process of any one of claims 1-15 and 17-19, wherein the mixture further comprises a base.

21. A process for preparing Compound A, or a salt thereof: 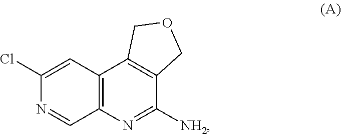 [SMILES: Nc1nc2cnc(Cl)cc2c2c1COC2]
comprising
(a) preparing a nickel(0) catalyst by exposing a mixture comprising a nickel(II) precatalyst, a phosphine, and an electrolyte in a solvent to a current to produce the nickel(0) catalyst; and
(b) contacting the nickel(0) catalyst with Compound B and Compound C in an admixing solvent to form Compound A or a salt thereof: 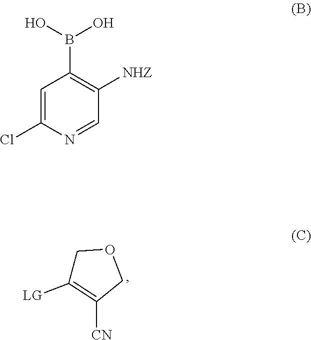
wherein Z is an amine protecting group and LG is a leaving group.

22. The process of claim 21, wherein the nickel(II) precatalyst is NiCl₂DME.

23. The process of claim 21 or 22, wherein the electrolyte comprises an alkylammonium salt, an imide salt, or a combination thereof.

24. The process of any one of claims 21-23, wherein the electrolyte comprises an alkali metal salt of NBu₄PF₆.

25. The process of claim 24, wherein the electrolyte comprises LiC₂F₆NO₄S₂.

26. The process of any one of claims 21-25, wherein solvent comprises acetonitrile, 2-methyltetrahydrofuran, tetrahydrofuran, dimethylacetamide, dimethylformamide, acetone, water, methanol, ethanol, isopropanol, toluene, dichloromethane, dimethylsulfoxide, N-methyl-2-pyrrolidone, or a combination thereof.

27. The process of any one of claims 21-26, wherein the phosphine comprises a monodentate phosphine ligand or a bidentate phosphine ligand.

28. The process of claim 27, wherein the phosphine comprises HP(nBu)₃-BF₄.

29. The process of any one of claims 21-28, wherein the current is applied across an anode and a cathode.

30. The process of any one of claims 21-29, wherein the mixture further comprises a base.

31. The process of any one of claims 21-30, wherein the carbamate is tert-butoxycarbonyl (Boc).

32. The process of any one of claims 21-31, wherein LG is a sulfonate ester, a sulfamate, or a halide.

33. The process of any one of claims 21-32, wherein the admixing solvent comprises acetonitrile, 2-methyltetrahydrofuran, dimethylacetamide, dimethylformamide, acetone, water, methanol, ethanol, isopropanol, toluene, DCM, DMSO, NMP, water, or a combination thereof.

34. The process of any one of claims 21-33, wherein the admixing solvent further comprises an inorganic salt.

35. The process of any one of claims 21-34, wherein the admixing is conducted in continuous mode.

36. A process for preparing Compound F 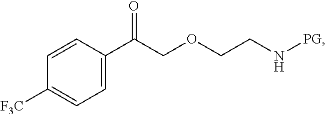
comprising
(a) reacting Compound H 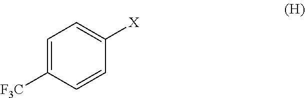
in a first solvent with an organolithium reagent to form a lithiated intermediate; and
(b) reacting the lithiated intermediate with Compound G 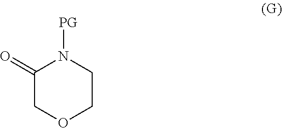
in a second solvent to form Compound F,
wherein X is halogen and PG is a protecting group.

37. The process of claim 36, wherein the organolithium reagent is an alkyllithium reagent.

38. The process of claim 36 or 37, wherein the first solvent comprises 2-methyltetrahydrofuran, tetrahydrofuran, 1,2-dimethoxyethane, 1,4-dioxane, tert-butyl methyl ether, cyclopentyl methyl ether, toluene, heptane, hexane, cyclohexane, or a combination thereof.

39. The process of any one of claims 36-38, wherein the second solvent comprises 2-methyltetrahydrofuran, tetrahydrofuran, 1,2-dimethoxyethane, 1,4-dioxane, tert-butyl methyl ether, cyclopentyl methyl ether, toluene, or a combination thereof.

40. The process of any one of claims 36-39, wherein the first and second solvents are the same.

41. The process of any one of claims 36-40, wherein X is F, Cl, Br, or I.

42. The process of any one of claims 36-41, wherein PG is a carbamate, a trialkylsilyl, a benzyl, a trityl or a sulfonyl protecting group.

43. The process of any one of claims 36-42, conducted in a continuous process apparatus or in semi-batch mode.

44. The process of any one of claims 36-43, further comprising using Compound F to prepare Compound I 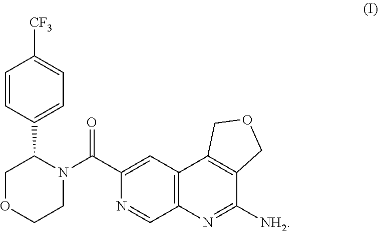 [SMILES: Nc1nc2cnc(C(=O)N3CCOC[C@@H]3c3ccc(C(F)(F)F)cc3)cc2c2c1COC2]
